(19) United States
(12) Patent Application Publication
(10) Pub. No.: US 2007/0270950 A1
(43) Pub. Date: Nov. 22, 2007

(54) Partial intervertebral implant and method of augmenting a disc surgery

(75) Inventor: Hai H. Trieu, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/356,757

(22) Filed: Feb. 17, 2006

(51) Int. Cl. A61F 2/44

(52) U.S. Cl. 623/17.11

(57) ABSTRACT

A partial nucleus implant is disclosed and can be installed within an intervertebral disc between an inferior vertebra and a superior vertebra proximate to a previously installed full nucleus implant. The partial nucleus implant can include a component that can be configured to be installed within a void between the full nucleus implant and an annulus fibrosis. Further, the component substantially secures the full nucleus implant in a desired position.

### FIELD OF THE DISCLOSURE

The present disclosure relates generally to orthopedics and spinal surgery. More specifically, the present disclosure relates to nucleus implants.

### BACKGROUND

In human anatomy, the spine is a generally flexible column that can take tensile and compressive loads. The spine also allows bending motion and provides a place of attachment for ribs, muscles and ligaments. Generally, the spine is divided into three sections: the cervical spine, the thoracic spine and the lumbar spine. The sections of the spine are made up of individual bones called vertebrae. Also, the vertebrae are separated by intervertebral discs, which are situated between adjacent vertebrae.

The intervertebral discs function as shock absorbers and as joints. Further, the intervertebral discs can absorb the compressive and tensile loads to which the spinal column may be subjected. At the same time, the intervertebral discs can allow adjacent vertebral bodies to move relative to each other a limited amount, particularly during bending, or flexure, of the spine. Thus, the intervertebral discs are under constant muscular and/or gravitational pressure and generally, the intervertebral discs are the first parts of the lumbar spine to show signs of “wear and tear”.

Facet joint degeneration is also common because the facet joints are in almost constant motion with the spine. In fact, facet joint degeneration and disc degeneration frequently occur together. Generally, although one may be the primary problem while the other is a secondary problem resulting from the altered mechanics of the spine, by the time surgical options are considered, both facet joint degeneration and disc degeneration typically have occurred. For example, the altered mechanics of the facet joints and/or intervertebral disc may cause spinal stenosis, degenerative spondylolisthesis, and degenerative scoliosis.

One surgical procedure for treating these conditions is spinal arthrodesis, i.e., spine fusion, which can be performed anteriorally, posteriorally, and/or laterally. The posterior procedures include in-situ fusion, posterior lateral instrumented fusion, transforaminal lumbar interbody fusion (“TLIF”) and posterior lumbar interbody fusion (“PLIF”). Solidly fusing a spinal segment to eliminate any motion at that level may alleviate the immediate symptoms, but for some patients maintaining motion may be beneficial. It is also known to surgically replace a degenerative disc or facet joint with an artificial disc or an artificial facet joint, respectively. Additionally, it is known to surgically remove nucleus pulposus material from within an intervertebral disc and replace the nucleus pulposus material with an artificial nucleus.

### BRIEF DESCRIPTION OF THE DRAWINGS

FIG. 1 is a lateral view of a portion of a vertebral column;

FIG. 2 is a lateral view of a pair of adjacent vertrebrae;

FIG. 3 is a top plan view of a vertebra;

FIG. 4 is a cross section view of an intervertebral disc;

FIG. 5 is a plan view of a first embodiment of a partial nucleus implant in a deflated position;

FIG. 6 is plan view of the first embodiment of the partial nucleus implant in an inflated position;

FIG. 7 is a plan view of a second embodiment of a partial nucleus implant in a deflated position;

FIG. 8 is a plan view of the second embodiment of the partial nucleus implant in an inflated position;

FIG. 9 is a plan view of a third embodiment of a partial nucleus implant in a deflated position;

FIG. 10 is a plan view of the third embodiment of the partial nucleus implant in an inflated position;

FIG. 11 is a cross-section view of a fourth embodiment of a partial nucleus implant in a deflated position;

FIG. 12 is a cross-section view of the fourth embodiment of the partial nucleus implant in an inflated position;

FIG. 13 is another cross-section view of the fourth embodiment of the partial nucleus implant in the inflated position;

FIG. 14 is a cross-section view of a fifth embodiment of the partial nucleus implant in a deflated position;

FIG. 15 is a cross-section view of the fifth embodiment of the partial nucleus implant in an inflated position;

FIG. 16 is a plan view of a sixth embodiment of a partial nucleus implant;

FIG. 17 is a flow chart of a method of revising a prior nucleus replacement; and

FIG. 18 is a flow chart of a method of augmenting a nucleus replacement surgery.

### DETAILED DESCRIPTION OF THE DRAWINGS

A partial nucleus implant is disclosed and can be installed within an intervertebral disc between an inferior vertebra and a superior vertebra proximate to a previously installed full nucleus implant. The partial nucleus implant can include a component that can be installed within a void between the full nucleus implant and an annulus fibrosis. Further, the component substantially secures the full nucleus implant in a desired position.

In another embodiment, a partial nucleus implant is disclosed and can be installed within an intervertebral disc between an inferior vertebra and a superior vertebra around a previously installed full nucleus implant. The partial nucleus implant can include a superior component that can include a superior surface that can be configured to engage a superior vertebra and an inferior surface that can be configured to engage a full nucleus implant. Further, the partial nucleus implant can include an inferior component that can include an inferior surface that can be configured to engage an inferior vertebra and a superior surface that can be configured to engage a full nucleus implant.

In yet another embodiment, a method of revising a prior nucleus replacement surgery is disclosed and can include examining a prior nucleus implant, examining an annulus fibrosis around the prior nucleus implant, and determining whether to reposition or replace the prior nucleus implant.

In still another embodiment, a method of revising a prior nucleus replacement surgery is disclosed and can include repositioning a prior nucleus implant and substantially securing the prior nucleus implant in a new position.

In yet another embodiment, a method of installing a nucleus implant is disclosed and includes installing a full nucleus implant. The method further includes installing a partial nucleus implant adjacent to the full nucleus implant.

### Description of Relevant Anatomy

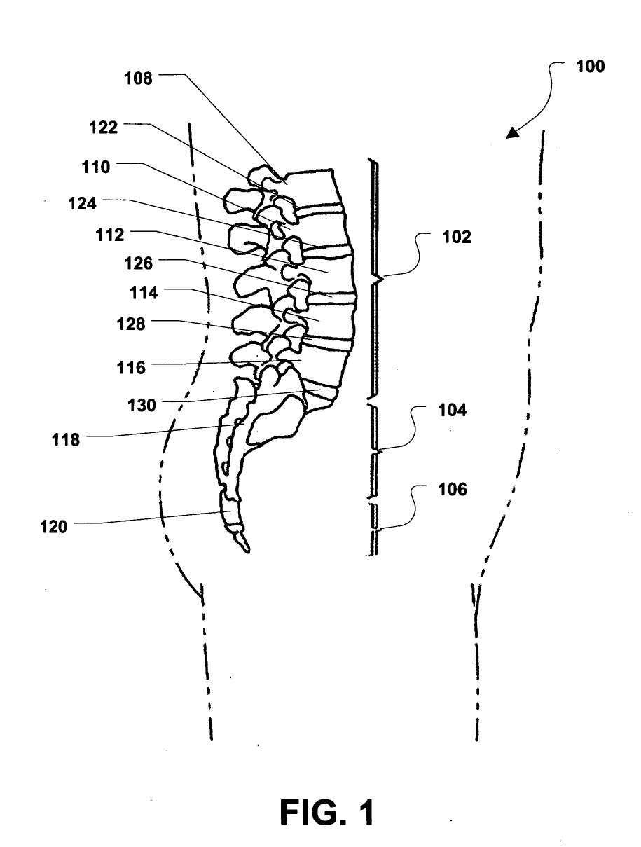

Referring initially to FIG. 1, a portion of a vertebral column, designated 100, is shown. As depicted, the vertebral column 100 includes a lumber region 102, a sacral region 104, and a coccygeal region 106. As is known in the art, the vertebral column 100 also includes a cervical region and a thoracic region. For clarity and ease of discussion, the cervical region and the thoracic region are not illustrated.

As shown in FIG. 1, the lumbar region 102 includes a first lumber vertebra 108, a second lumbar vertebra 110, a third lumbar vertebra 112, a fourth lumbar vertebra 114, and a fifth lumbar vertebra 116. The sacral region 104 includes a sacrum 118. Further, the coccygeal region 106 includes a coccyx 120.

As depicted in FIG. 1, a first intervertebral lumbar disc 122 is disposed between the first lumber vertebra 108 and the second lumbar vertebra 110. A second intervertebral lumbar disc 124 is disposed between the second lumbar vertebra 110 and the third lumbar vertebra 112. A third intervertebral lumbar disc 126 is disposed between the third lumbar vertebra 112 and the fourth lumbar vertebra 114. Further, a fourth intervertebral lumbar disc 128 is disposed between the fourth lumbar vertebra 114 and the fifth lumbar vertebra 116. Additionally, a fifth intervertebral lumbar disc 130 is disposed between the fifth lumbar vertebra 116 and the sacrum 118.

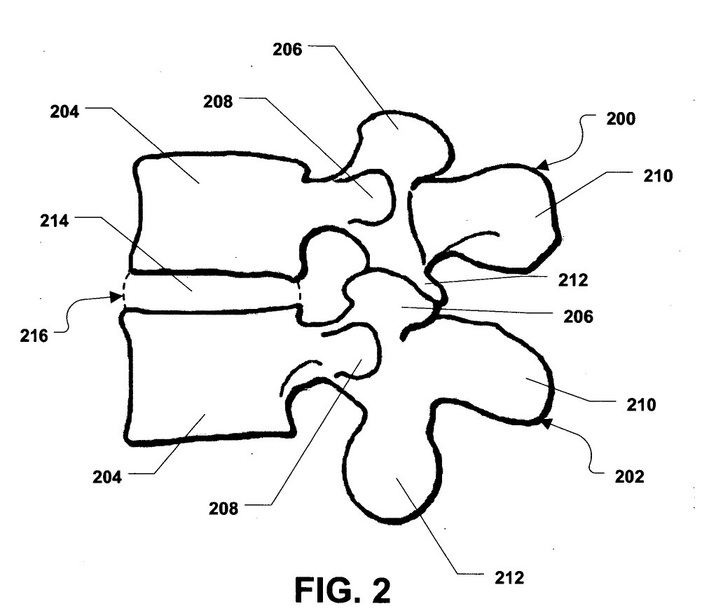

FIG. 2 depicts a detailed lateral view of two adjacent vertebrae, e.g., two of the lumbar vertebra 108, 110, 112, 114, 116 shown in FIG. 1. FIG. 2 illustrates a superior vertebra 200 and an inferior vertebra 202. As shown, each vertebra 200, 202 includes a vertebral body 204, a superior articular process 206, a transverse process 208, a spinous process 210 and an inferior articular process 212. FIG. 2 further depicts an intervertebral space 214 that can be established between the superior vertebra 200 and the inferior vertebra 202 by removing an intervertebral disc 216 (shown in dashed lines).

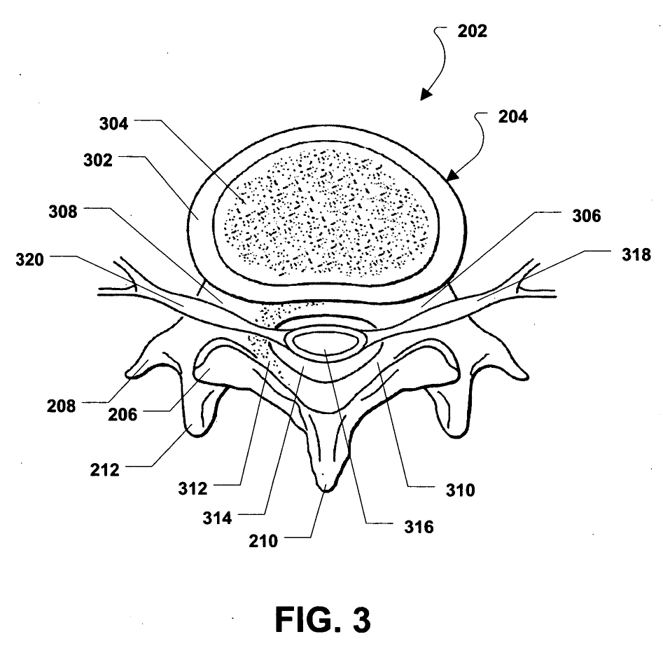

Referring to FIG. 3, a vertebra, e.g., the inferior vertebra 202 (FIG. 2), is illustrated. As shown, the vertebral body 204 of the inferior vertebra 202 includes a cortical rim 302 composed of cortical bone. Also, the vertebral body 204 includes cancellous bone 304 within the cortical rim 302. The cortical rim 302 is often referred to as the apophyseal rim or apophyseal ring. Further, the cancellous bone 304 is softer and weaker than the cortical bone of the cortical rim 302.

As illustrated in FIG. 3, the inferior vertebra 202 further includes a first pedicle 306, a second pedicle 308, a first lamina 310, and a second lamina 312. Further, a vertebral foramen 314 is established within the inferior vertebra 202. A spinal cord 316 passes through the vertebral foramen 314. Moreover, a first nerve root 318 and a second nerve root 320 extend from the spinal cord 316.

It is well known in the art that the vertebrae that make up the vertebral column have slightly different appearances as they range from the cervical region to the lumbar region of the vertebral column. However, all of the vertebrae, except the first and second cervical vertebrae, have the same basic structures, e.g., those structures described above in conjunction with FIG. 2 and FIG. 3. The first and second cervical vertebrae are structurally different than the rest of the vertebrae in order to support a skull.

Referring now to FIG. 4, an intervertebral disc is shown and is generally designated 400. The intervertebral disc 400 is made up of two components: the annulus fibrosis 402 and the nucleus pulposus 404. The annulus fibrosis 402 is the outer portion of the intervertebral disc 400, and the annulus fibrosis 402 includes a plurality of lamellae 406. The lamellae 406 are layers of collagen and proteins. Each lamella 406 includes fibers that slant at 30-degree angles, and the fibers of each lamella 406 run in a direction opposite the adjacent layers. Accordingly, the annulus fibrosis 402 is a structure that is exceptionally strong, yet extremely flexible.

The nucleus pulposus 404 is the inner gel material that is surrounded by the annulus fibrosis 402. It makes up about forty percent (40%) of the intervertebral disc 400 by weight. Moreover, the nucleus pulposus 404 can be considered a ball-like gel that is contained within the lamellae 406. The nucleus pulposus 404 includes loose collagen fibers, water, and proteins. The water content of the nucleus pulposus 404 is about ninety percent (90%) by weight at birth and decreases to about seventy percent by weight (70%) by the fifth decade.

Injury or aging of the annulus fibrosis 402 may allow the nucleus pulposus 404 to be squeezed through the annulus fibers either partially, causing the disc to bulge, or completely, allowing the disc material to escape the intervertebral disc 400. The bulging disc or nucleus material may compress the nerves or spinal cord, causing pain. Accordingly, the nucleus pulposus 404 can be removed and replaced with an artificial nucleus.

### DESCRIPTION OF A FIRST EMBODIMENT

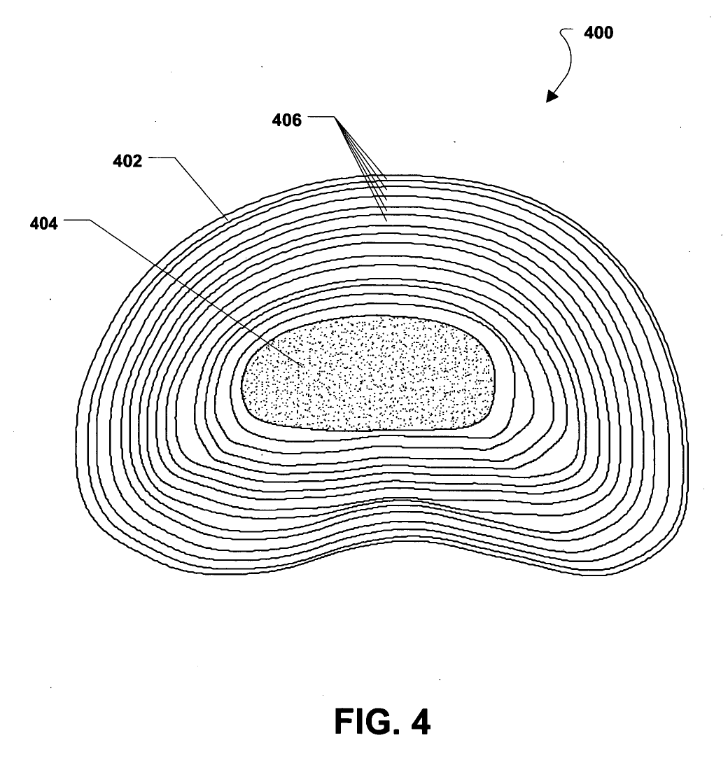

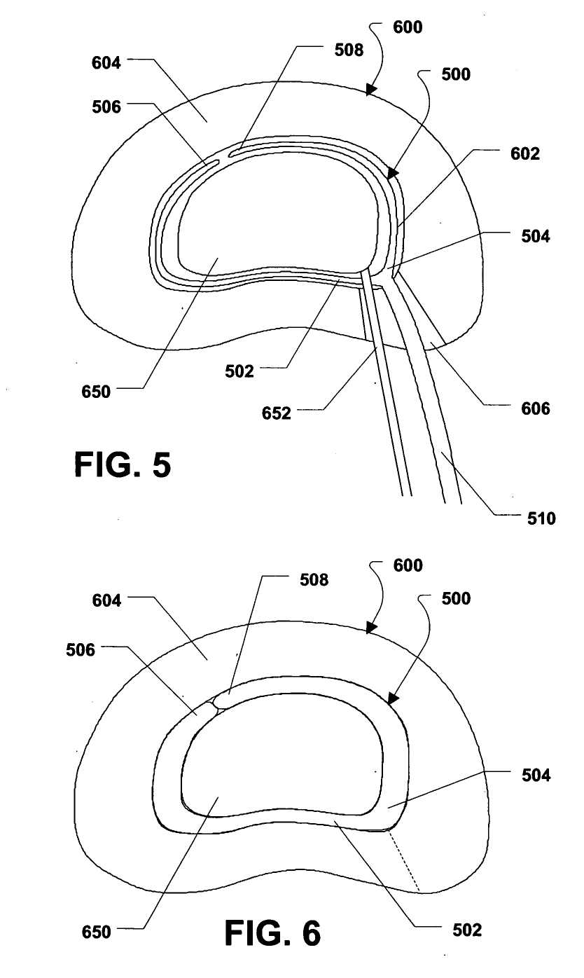

Referring to FIG. 5 and FIG. 6, an embodiment of a partial disc implant, i.e., a partial nucleus implant, is shown and is designated 500. As shown, the partial nucleus implant 500 includes an expandable component 502 that has a proximal end 504, a first distal end 506, and a second distal end 508. Further, the partial nucleus implant 500 includes an injection tube 510 that extends from the proximal end 504 of the expandable component 502. In a particular embodiment, the expandable component 502 of the partial nucleus implant 500 is expandable from a deflated position, shown in FIG. 5, to one of a plurality of inflated positions, shown in FIG. 6, up to a maximum inflated position. Further, after the expandable component 502 is inflated, or otherwise expanded, the injection tube 508 can be removed, as depicted in FIG. 6.

FIG. 5 and FIG. 6 indicate that the partial nucleus implant 500 can be implanted within an intervertebral disc 600. More specifically, the expandable component 502 of the partial nucleus implant 500 can be implanted within an intervertebral disc space 602 established within the annulus fibrosis 604 of the intervertebral disc 600. The intervertebral disc space 602 can be established by removing the nucleus pulposus (not shown) from within the annulus fibrosis 602.

Further, in a particular embodiment, the expandable component 502 of the partial nucleus implant 500 can be implanted within the intervertebral disc space 602 around a full nucleus implant 650 that was implanted within the intervertebral disc space 602 during a prior nucleus replacement surgery. Accordingly, the expandable component 502 of the partial nucleus implant 500 can be implanted within a void, or space, between the full nucleus implant 650 and the annulus fibrosis 604. Accordingly, in the event that the full nucleus implant 650 is undersized, the expandable component 502 of the partial nucleus implant 500 can be installed around the full nucleus implant 650 during a revision surgery in order to reposition the full nucleus implant and prevent the full nucleus implant 650 from moving with the annulus fibrosis 604.

As shown in FIG. 5, a nucleus implant holder 652 can be used to engage the full nucleus implant 650 and position the full nucleus implant 650 while the expandable component 502 of the partial nucleus implant 500 is inserted within the annulus fibrosis 604 around the full nucleus implant 650 and while the expandable component 502 is expanded, or inflated, around the full nucleus implant 650. In a particular embodiment, the partial nucleus implant 500 can include a self-sealing valve (not shown) within the proximal end 504 of the expandable component 502 that can prevent the expandable component 502 from leaking material after the expandable component 502 is inflated and the injection tube 502 is removed.

Further, as shown in FIG. 6, the distal ends 506, 508 of the expandable component 502 can be positioned such that a contiguous portion of the expandable component 502 spans the incision made in the annulus fibrosis. FIG. 6 depicts the incision as a dashed line. As such, the expandable component 502 may minimize any risk that the full nucleus implant 650 re-open the incision while the patient is healing.

In a particular embodiment, the expandable component 502 of the partial nucleus implant 500 can be inflated with one or more injectable biocompatible materials that remain elastic after curing. Further, the injectable biocompatible materials can include polymer materials that remain elastic after curing. Also, the injectable biocompatible materials can include ceramics.

For example, the polymer materials can include polyurethane, polyolefin, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefin, and flouropolyolefin.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

In a particular embodiment, the ceramics can include calcium phosphate, hydroxyapatite, calcium sulfate, bioactive glass, or a combination thereof.

In an alternative embodiment, the injectable biocompatible materials can include one or more fluids such as sterile water, saline, or sterile air. In alternative embodiments, the expandable component 502 of the partial nucleus implant 500 can be inflated with one or more of the following: fibroblasts, chondroblasts, differentiated stem cells or other biologic factor which would create a motion limiting tissue when injected into a bioresorbable motion limiting scaffold.

In another alternative embodiment, the partial nucleus implant 500 can be a solid implant that is formed external to the patient and then, implanted within an intervertebral disc space within an annulus fibrosis. The solid partial nucleus implant can have substantially the same shape as the expanded partial nucleus implant 500 depicted in FIG. 6. Further, the solid partial nucleus implant 500 can be made from one or more biocompatible materials that remain elastic after curing. In a particular embodiment, the biocompatible materials can include polymer materials. The polymer materials can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefm, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

In a particular embodiment, the partial nucleus implant 500 can be installed using a posterior surgical approach, as shown. Further, the partial nucleus implant 500 can be installed through a posterior incision 606 made within the annulus fibrosis 604 of the intervertebral disc 600. Alternatively, the partial nucleus implant 500 can be installed using an anterior surgical approach or a lateral surgical approach.

### DESCRIPTION OF A SECOND EMBODIMENT

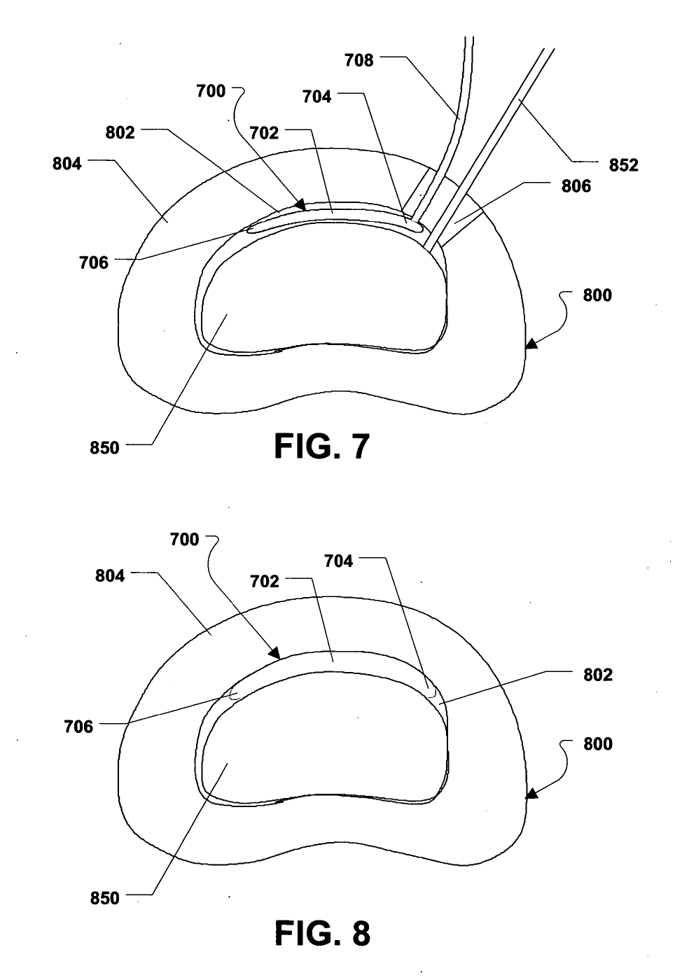

Referring to FIG. 7 and FIG. 8, a second embodiment of a partial nucleus implant is shown and is designated 700. As shown, the partial nucleus implant 700 includes an expandable component 702 that has a proximal end 704 and a distal end 706. Further, the partial nucleus implant 700 includes an injection tube 708 that extends from the proximal end 704 of the expandable component 702. In a particular embodiment, the expandable component 702 of the partial nucleus implant 700 is expandable from a deflated position, shown in FIG. 7, to one of a plurality of inflated positions, shown in FIG. 8, up to a maximum inflated position. Further, after the expandable component 702 is inflated, or otherwise expanded, the injection tube 708 can be removed, as depicted in FIG. 8.

FIG. 7 and FIG. 8 indicate that the partial nucleus implant 700 can be implanted within an intervertebral disc 800. More specifically, the expandable component 702 of the partial nucleus implant 700 can be implanted within an intervertebral disc space 802 established within the annulus fibrosis 804 of the intervertebral disc 800. The intervertebral disc space 802 can be established by removing the nucleus pulposus (not shown) from within the annulus fibrosis 802.

Further, in a particular embodiment, the expandable component 702 of the partial nucleus implant 700 can be implanted within the intervertebral disc space 802 anterior to a full nucleus implant 850 that was implanted within the intervertebral disc space 802 during a prior nucleus replacement surgery. Accordingly, the expandable component 702 of the partial nucleus implant 700 can be implanted within a void, or space, between the full nucleus implant 850 and the annulus fibrosis 804. Accordingly, in the event that the full nucleus implant 850 is undersized, the expandable component 702 of the partial nucleus implant 700 can be installed adjacent to the full nucleus implant 850 during a revision surgery in order to reposition the full nucleus implant and prevent the full nucleus implant 850 from moving with the annulus fibrosis 804.

As shown in FIG. 7, a nucleus implant holder 852 can be used to engage the full nucleus implant 850 and position the full nucleus implant 850 while the expandable component 702 of the partial nucleus implant 700 is inserted within the annulus fibrosis 804 anterior to the full nucleus implant 850 and while the expandable component 702 is expanded, or inflated, adjacent to the full nucleus implant 850. In a particular embodiment, the partial nucleus implant 700 can include a self-sealing valve (not shown) within the proximal end 704 of the expandable component 702 that can prevent the expandable component 702 from leaking material after the expandable component 702 is inflated and the injection tube 702 is removed.

In a particular embodiment, the expandable component 702 of the partial nucleus implant 700 can be inflated with one or more injectable biocompatible materials that remain elastic after curing. Further, the injectable biocompatible materials can include polymer materials that remain elastic after curing. Also, the injectable biocompatible materials can include ceramics.

For example, the polymer materials can include polyurethane, polyolefin, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefin, and flouropolyolefin.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

In a particular embodiment, the ceramics can include calcium phosphate, hydroxyapatite, calcium sulfate, bioactive glass, or a combination thereof.

In an alternative embodiment, the injectable biocompatible materials can include one or more fluids such as sterile water, saline, or sterile air. In alternative embodiments, the expandable component 702 of the partial nucleus implant 700 can be inflated with one or more of the following: fibroblasts, chondroblasts, differentiated stem cells or other biologic factor which would create a motion limiting tissue when injected into a bioresorbable motion limiting scaffold.

In another alternative embodiment, the partial nucleus implant 700 can be a solid implant that is formed external to the patient and then, implanted within an intervertebral disc space within an annulus fibrosis. The solid partial nucleus implant can have substantially the same shape as the expanded partial nucleus implant 700 depicted in FIG. 8. Further, the solid partial nucleus implant 700 that is made from one or more biocompatible materials that remain elastic after curing. In a particular embodiment, the biocompatible materials can include polymer materials. The polymer materials can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

In a particular embodiment, the partial nucleus implant 700 can be installed using an anterior surgical approach, as shown. Further, the partial nucleus implant 700 can be installed through an anterior incision 806 made within the annulus fibrosis 804 of the intervertebral disc 800. Alternatively, the partial nucleus implant 700 can be installed using a posterior surgical approach or a lateral surgical approach.

### DESCRIPTION OF A THIRD EMBODIMENT

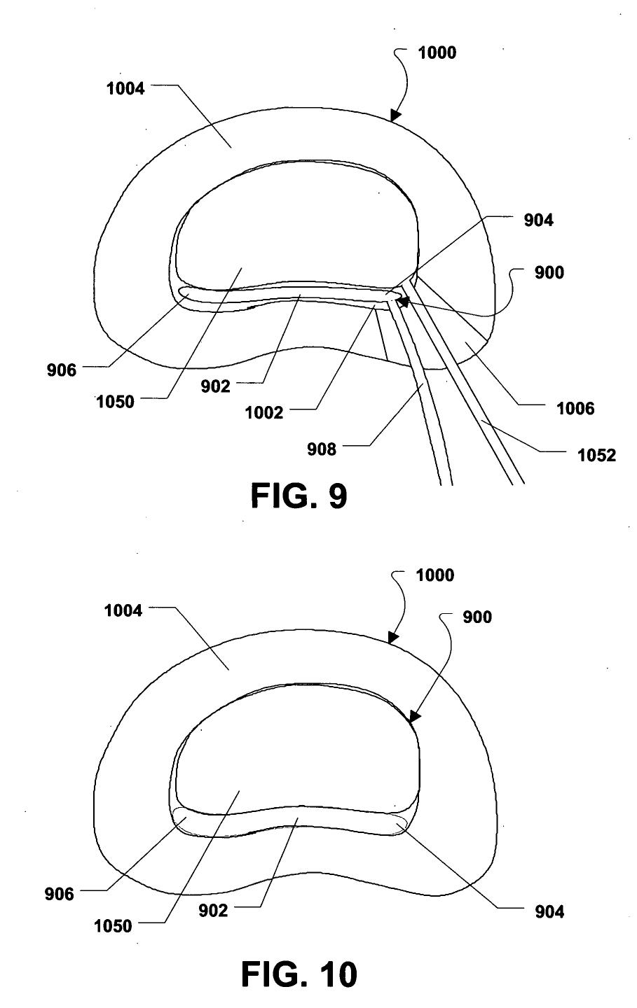

Referring to FIG. 9 and FIG. 10, a third embodiment of a partial nucleus implant is shown and is designated 900. As shown, the partial nucleus implant 900 includes an expandable component 902 that has a proximal end 904 and a distal end 906. Further, the partial nucleus implant 900 includes an injection tube 908 that extends from the proximal end 904 of the expandable component 902. In a particular embodiment, the expandable component 902 of the partial nucleus implant 900 is expandable from a deflated position, shown in FIG. 9, to one of a plurality of inflated positions, shown in FIG. 10, up to a maximum inflated position. Further, after the expandable component 902 is inflated, or otherwise expanded, the injection tube 908 can be removed, as depicted in FIG. 10.

FIG. 9 and FIG. 10 indicate that the partial nucleus implant 900 can be implanted within an intervertebral disc 1000. More specifically, the expandable component 902 of the partial nucleus implant 900 can be implanted within an intervertebral disc space 1002 established within the annulus fibrosis 1004 of the intervertebral disc 1000. The intervertebral disc space 1002 can be established by removing the nucleus pulposus (not shown) from within the annulus fibrosis 1002.

Further, in a particular embodiment, the expandable component 902 of the partial nucleus implant 900 can be implanted within the intervertebral disc space 1002 posterior to a full nucleus implant 1050 that was implanted within the intervertebral disc space 1002 during a prior nucleus replacement surgery. Accordingly, the expandable component 902 of the partial nucleus implant 900 can be implanted within a void, or space, between the full nucleus implant 1050 and the annulus fibrosis 1004. Accordingly, in the event that the full nucleus implant 1050 is undersized, the expandable component 902 of the partial nucleus implant 900 can be installed adjacent to the full nucleus implant 1050 during a revision surgery in order to reposition the full nucleus implant and prevent the full nucleus implant 1050 from moving with the annulus fibrosis 1004.

As shown in FIG. 9, a nucleus implant holder 1052 can be used to engage the full nucleus implant 1050 and position the full nucleus implant 1050 while the expandable component 902 of the partial nucleus implant 900 is inserted within the annulus fibrosis 1004 posterior to the full nucleus implant 1050 and while the expandable component 902 is expanded, or inflated, adjacent to the full nucleus implant 1050. In a particular embodiment, the partial nucleus implant 900 can include a self-sealing valve (not shown) within the proximal end 904 of the expandable component 902 that can prevent the expandable component 902 from leaking material after the expandable component 902 is inflated and the injection tube 902 is removed.

In a particular embodiment, the expandable component 902 of the partial nucleus implant 900 can be inflated with one or more injectable biocompatible materials that remain elastic after curing. Further, the injectable biocompatible materials can include polymer materials that remain elastic after curing. Also, the injectable biocompatible materials can include ceramics.

For example, the polymer materials can include polyurethane, polyolefin, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefin, and flouropolyolefin.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

In a particular embodiment, the ceramics can include calcium phosphate, hydroxyapatite, calcium sulfate, bioactive glass, or a combination thereof.

In an alternative embodiment, the injectable biocompatible materials can include one or more fluids such as sterile water, saline, or sterile air. In alternative embodiments, the expandable component 902 of the partial nucleus implant 900 can be inflated with one or more of the following: fibroblasts, chondroblasts, differentiated stem cells or other biologic factor which would create a motion limiting tissue when injected into a bioresorbable motion limiting scaffold.

In another alternative embodiment, the partial nucleus implant 900 can be a solid implant that is formed external to the patient and then, implanted within an intervertebral disc space within an annulus fibrosis. The solid partial nucleus implant can have substantially the same shape as the expanded partial nucleus implant 900 depicted in FIG. 10. Further, the solid partial nucleus implant 900 can be made from one or more biocompatible materials that remain elastic after curing. In a particular embodiment, the biocompatible materials can include polymer materials. The polymer materials can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

In a particular embodiment, the partial nucleus implant 900 can be installed using a posterior surgical approach, as shown. Further, the partial nucleus implant 900 can be installed through a posterior incision 1006 made within the annulus fibrosis 1004 of the intervertebral disc 1000. Alternatively, the partial nucleus implant 900 can be installed using an anterior surgical approach or a lateral surgical approach.

### DESCRIPTION OF A FOURTH EMBODIMENT

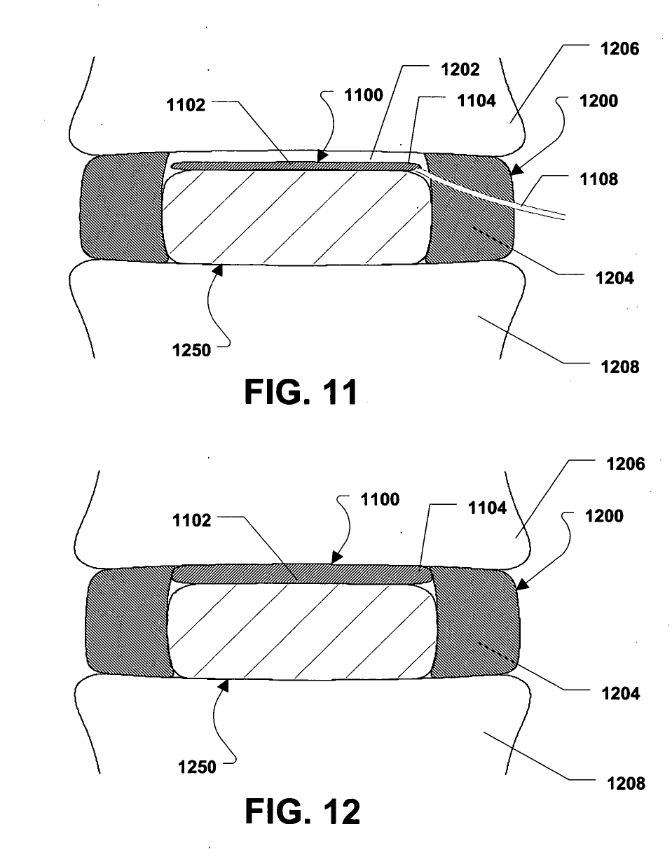

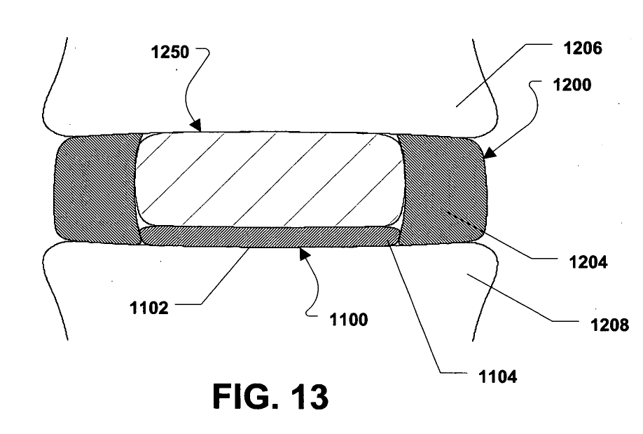

Referring to FIG. 11, FIG. 12, and FIG. 13, a fourth embodiment of a partial nucleus implant is shown and is designated 1100. As shown, the partial nucleus implant 1100 includes an expandable component 1102 having a periphery 1104. Further, the partial nucleus implant 1100 includes an injection tube 1108 that extends from the periphery 1104 of the expandable component 1102. In a particular embodiment, the expandable component 1102 of the partial nucleus implant 1100 is expandable from a deflated position, shown in FIG. 11, to one of a plurality of inflated positions, shown in FIG. 12, up to a maximum inflated position. Further, after the expandable component 1102 is inflated, or otherwise expanded, the injection tube 1108 can be removed, as depicted in FIG. 12.

FIG. 11 and FIG. 12 indicate that the partial nucleus implant 1100 can be implanted within an intervertebral disc 1200. More specifically, the expandable component 1102 of the partial nucleus implant 1100 can be implanted within an intervertebral disc space 1202 established within the annulus fibrosis 1204 of the intervertebral disc 1200. The intervertebral disc space 1202 can be established by removing the nucleus pulposus (not shown) from within the annulus fibrosis 1202.

Further, in a particular embodiment, the expandable component 1102 of the partial nucleus implant 1100 can be implanted within the intervertebral disc space 1202 superior to, or above, a full nucleus implant 1250 that was implanted within the intervertebral disc space 1202 during a prior nucleus replacement surgery. Accordingly, the expandable component 1102 of the partial nucleus implant 1100 can be implanted within a void, or space, between the full nucleus implant 1250 and a superior vertebra 1206. Accordingly, in the event that the full nucleus implant 1250 is undersized, e.g., too short, the expandable component 1102 of the partial nucleus implant 1100 can be installed on top of the full nucleus implant 1250 during a revision surgery in order to reposition the full nucleus implant and prevent the full nucleus implant 1250 from moving with the annulus fibrosis 1204. Alternatively, the partial nucleus implant 1100 can be installed underneath the full nucleus implant 1250, e.g., between the full nucleus implant 1250 and an inferior vertebra 1208, as shown in FIG. 13.

In a particular embodiment, the partial nucleus implant 1100 can include a self-sealing valve (not shown) within the periphery 1104 of the expandable component 1102 that can prevent the expandable component 1102 from leaking material after the expandable component 1102 is inflated and the injection tube 1102 is removed.

In a particular embodiment, the expandable component 1102 of the partial nucleus implant 1100 can be inflated with one or more injectable biocompatible materials that remain elastic after curing. Further, the injectable biocompatible materials can include polymer materials that remain elastic after curing. Also, the injectable biocompatible materials can include ceramics.

For example, the polymer materials can include polyurethane, polyolefin, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefin, and flouropolyolefm.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

In a particular embodiment, the ceramics can include calcium phosphate, hydroxyapatite, calcium sulfate, bioactive glass, or a combination thereof.

In an alternative embodiment, the injectable biocompatible materials can include one or more fluids such as sterile water, saline, or sterile air. In alternative embodiments, the expandable component 1102 of the partial nucleus implant 1100 can be inflated with one or more of the following: fibroblasts, chondroblasts, differentiated stem cells or other biologic factor which would create a motion limiting tissue when injected into a bioresorbable motion limiting scaffold.

In another alternative embodiment, the partial nucleus implant 1100 can be a solid implant that is formed external to the patient and then, implanted within an intervertebral disc space within an annulus fibrosis. The solid partial nucleus implant can have substantially the same shape as the expanded partial nucleus implant 1100 depicted in FIG. 12 or FIG. 13. Further, the solid partial nucleus implant 1100 that is made from one or more biocompatible materials that remain elastic after curing. In a particular embodiment, the biocompatible materials can include polymer materials. The polymer materials can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

In a particular embodiment, the partial nucleus implant 1100 can be installed using a posterior surgical approach, an anterior surgical approach, a lateral surgical approach, or any other surgical approach well known in the art.

### DESCRIPTION OF A FIFTH EMBODIMENT

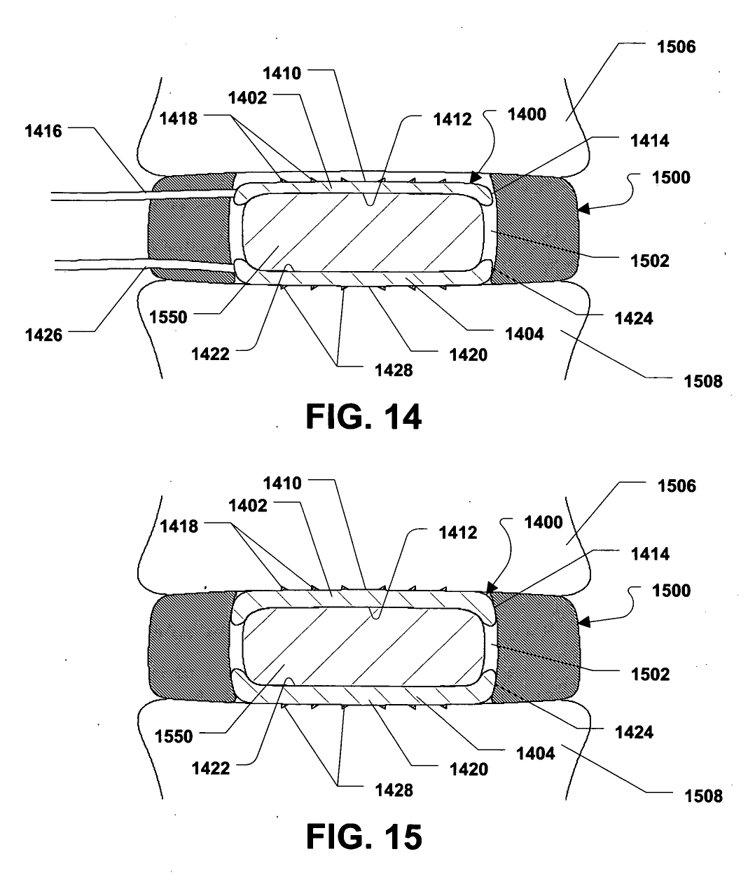

Referring to FIG. 14 and FIG. 15, a fifth embodiment of a partial nucleus implant is shown and is designated 1400. As shown, the partial nucleus implant 1400 can include a superior expandable component 1402 and an inferior expandable component 1404.

As shown, the superior expandable component 1402 can include a generally convex superior surface 1410, a generally concave inferior surface 1412 and a periphery 1414. Further, the partial nucleus implant 1400 includes a superior injection tube 1416 that extends from the periphery 1414 of the superior expandable component 1402. Additionally, a plurality of superior teeth 1418 can extend from the superior surface 1410 of the superior expandable component 1402.

As shown, in a particular embodiment, the superior teeth 1418 are generally saw-tooth, or triangle, shaped. Further, the superior teeth 1418 are designed to engage cancellous bone, or-cortical bone, of a superior vertebra. Additionally, the superior teeth 1418 can prevent the superior expandable component 1402 from moving with respect to a superior vertebra after the partial nucleus implant 1400 is installed as described herein.

In a particular embodiment, the superior teeth 1418 can include other projections such as spikes, pins, blades, or a combination thereof that have any cross-sectional geometry.

Further, the inferior expandable component 1404 can include a generally convex inferior surface 1420, a generally concave superior surface 1422 and a periphery 1424. Further, the partial nucleus implant 1500 includes an inferior injection tube 1426 that extends from the periphery 1424 of the inferior expandable component 1404. Additionally, a plurality of inferior teeth 1428 can extend from the inferior surface 1420 of the inferior expandable component 1404.

As shown, in a particular embodiment, the inferior teeth 1428 are generally saw-tooth, or triangle, shaped. Further, the inferior teeth 1428 are designed to engage cancellous bone, or cortical bone, of an inferior vertebra. Additionally, the inferior teeth 1428 can prevent the inferior expandable component 1404 from moving with respect to an inferior vertebra after the partial nucleus implant 1400 is installed as described herein.

In a particular embodiment, the inferior teeth 1428 can include other projections such as spikes, pins, blades, or a combination thereof that have any cross-sectional geometry.

In a particular embodiment, each of the expandable components 1402, 1404 of the partial nucleus implant 1400 is expandable from a deflated position, shown in FIG. 14, to one of a plurality of inflated positions, shown in FIG. 15, up to a maximum inflated position. Further, after each expandable component 1402, 1404 is inflated, or otherwise expanded, the corresponding injection tube 1416, 1426 can be removed, as depicted in FIG. 15.

FIG. 14 and FIG. 15 indicate that the partial nucleus implant 1400 can be implanted within an intervertebral disc 1500. More specifically, the expandable component 1402 of the partial nucleus implant 1400 can be implanted within an intervertebral disc space 1502 established within the annulus fibrosis 1504 of the intervertebral disc 1500. The intervertebral disc space 1502 can be established by removing the nucleus pulposus (not shown) from within the annulus fibrosis 1502.

Further, in a particular embodiment, the superior expandable component 1402 of the partial nucleus implant 1400 can be implanted within the intervertebral disc space 1502 superior to, or above, a full nucleus implant 1550 that was implanted within the intervertebral disc space 1502 during a prior nucleus replacement surgery. Also, the inferior expandable component 1404 can be implanted within the intervertebral disc space 1502 inferior to, or below, the full nucleus implant 1550. Accordingly, the expandable components 1402, 1404 of the partial nucleus implant 1400 can be implanted between the full nucleus implant 1550 and a superior vertebra 1506 and between the full nucleus implant 1550 and an inferior vertebra 1508.

As depicted in FIG. 15, when the expandable components 1402, 1404 are properly inflated, or expanded, the full nucleus implant 1550 is cupped between the inferior surface 1412 of the superior expandable component 1402 and the superior surface 1422 of the inferior expandable component 1404. Further, the superior teeth 1418 can engage the superior vertebra 1506 and the inferior teeth 1428 can engage the inferior vertebra 1508. As such, in the event that the full nucleus implant 1550 is undersized the partial nucleus implant 1400 can be installed around the full nucleus implant 1550 during a revision surgery in order to reposition the full nucleus implant and prevent the full nucleus implant 1550 from moving with the annulus fibrosis 1504. In alternative embodiments, the partial nucleus implant 1400 may only include the superior expandable component 1402 and associated elements or the inferior expandable component 1404 and associated elements.

In a particular embodiment, each expandable component 1402, 1404 can include a self-sealing valve (not shown) that can prevent each expandable component 1402, 1404 from leaking material after the expandable components 1402, 1404 are inflated and the corresponding injection tubes 1416, 1426 are removed.

In a particular embodiment, the expandable components 1402, 1404 of the partial nucleus implant 1400 can be inflated with one or more injectable biocompatible materials that remain elastic after curing. Further, the injectable biocompatible materials can include polymer materials that remain elastic after curing. Also, the injectable biocompatible materials can include ceramics.

For example, the polymer materials can include polyurethane, polyolefm, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefin, and flouropolyolefin.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

In a particular embodiment, the ceramics can include calcium phosphate, hydroxyapatite, calcium sulfate, bioactive glass, or a combination thereof.

In an alternative embodiment, the injectable biocompatible materials can include one or more fluids such as sterile water, saline, or sterile air. In alternative embodiments, the expandable components 1402, 1404 of the partial nucleus implant 1400 can be inflated with one or more of the following: fibroblasts, chondroblasts, differentiated stem cells or other biologic factor which would create a motion limiting tissue when injected into a bioresorbable motion limiting scaffold.

In another alternative embodiment, the partial nucleus implant 1400 can be a solid implant that is formed external to the patient and then, implanted within an intervertebral disc space within an annulus fibrosis. The solid partial nucleus implant can have substantially the same shape as the expanded partial nucleus implant 1400 depicted in FIG. 15. Further, the solid partial nucleus implant 1400 that is made from one or more biocompatible materials that remain elastic after curing. In a particular embodiment, the biocompatible materials can include polymer materials. The polymer materials can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

In a particular embodiment, the partial nucleus implant 1400 can be installed using a posterior surgical approach, an anterior surgical approach, a lateral surgical approach, or any other surgical approach well known in the art.

### DESCRIPTION OF A SIXTH EMBODIMENT

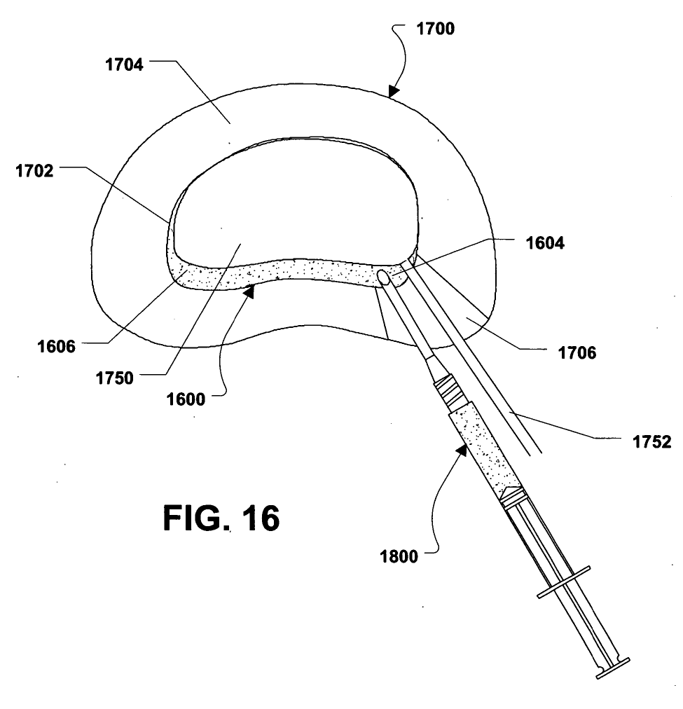

Referring to FIG. 16, an embodiment of a partial nucleus implant is shown and is designated 1600. As shown, the partial nucleus implant 1600 includes a proximal end 1604 and a distal end 1606.

FIG. 16 indicates that the partial nucleus implant 1600 can be implanted within an intervertebral disc 1700. More specifically, the partial nucleus implant 1600 can be injected into an intervertebral disc space 1702 established within the annulus fibrosis 1704 of the intervertebral disc 1700. For example, the partial nucleus implant 1600 can be injected into the intervertebral disc space 1702 using a syringe 1800.

In a particular embodiment, the partial nucleus implant 1600 can be made from one or more biocompatible materials. In a particular embodiment, the biocompatible materials can include one or more curable biomaterials. The curable biomaterials can include any natural or synthetic materials with or without adhesive properties that can undergo phase transformation from a flowable to a non-flowable state due to gelation, crystallization, crosslinking, solidification, etc. Further, the curable biomaterials can be resorbable, non-resorbable, compliant, semi-compliant, rigid, elastic, semi-elastic, inelastic, or a combination thereof.

The curable biomaterials can include polymer materials, hydrogels, proteins, and polysaccharides. The polymer materials can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

For example, the polymer materials can include polyurethane, polyolefin, silicone, silicone polyurethane copolymers, polymethylmethacrylate, epoxy, cyanoacrylate, hydrogels, resorbable polymers, or a combination thereof. Further, the polyolefin materials can include polypropylene, polyethylene, halogenated polyolefin, and flouropolyolefin.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

The proteins can include collagen, silk, elastin, keratin, albumin, gelatin, de-mineralized bone matrix, fibrin, or a combination thereof. Further, the polysaccharides can include glycosaminoglycan (GAG), hyaluronic acid (HA), carboxymethylcellulose (CMC), or a combination thereof

In a particular embodiment, the partial nucleus implant 1600 can include one or more additives that can be injected therewith. For example, the additives can include water, solvents, radiocontrast media, drugs, cellular matters, biological factors, or a combination thereof. In a particular embodiment, the drugs can include antibiotics, analgesics, anti-inflammatory drugs, anti-TNF-alpha, steroids, or a combination thereof. Further, the cellular matters can include bone marrow derived stem cells, lipo derived stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, or a combination thereof.

In a particular embodiment, the partial nucleus implant 1600 can include a reinforcing structure to supplement or reinforce the partial nucleus implant 1600. The reinforcing structure can be a fibrous structure, a mesh structure, a woven structure, a braided structure, or a combination thereof that is disposed at least partially within or at least partially around the partial nucleus implant 1600.

In a particular embodiment, the partial nucleus implant 1600 can cure naturally, i.e., under ambient conditions, in situ. Alternatively, the partial nucleus implant 1600 can be cured in situ using an energy source. For example, the energy source can be a light source that emits visible light, infrared (IR) light, or ultra-violet (UV) light. Further, the energy source can be a heating device, a radiation device, or other mechanical device. In a particular embodiment, the intervertebral disc space 1702 can be established by removing the nucleus pulposus (not shown) from within the annulus fibrosis 1702.

Further, in a particular embodiment, the partial nucleus implant 1600 can be injected, or otherwise implanted, within the intervertebral disc space 1702 adjacent to a full nucleus implant 1750 that was implanted within the intervertebral disc space 1702 during a prior nucleus replacement surgery or during the same surgery. Accordingly, the partial nucleus implant 1600 can be injected, or otherwise implanted, within a void, or space, between the full nucleus implant 1750 and the annulus fibrosis 1704. Moreover, in its flowable state, the partial nucleus implant 1600 can take the shape of the void before it cures and substantially fill the void. Accordingly, in the event that the full nucleus implant 1750 is undersized, the partial nucleus implant 1600 can be installed adjacent to, or at least partially around, the full nucleus implant 1750 during a revision surgery in order to reposition the full nucleus implant and prevent the full nucleus implant 1750 from moving with the annulus fibrosis 1704. Further, the partial nucleus implant 1600 can be injected, or otherwise implanted, around, superior to, inferior to, anterior to, posterior to, laterally adjacent to, or otherwise adjacent to the full nucleus implant 1750.

As shown in FIG. 16, a nucleus implant holder 1752 can be used to engage the full nucleus implant 1750 and position the full nucleus implant 1750 while the partial nucleus implant 1600 is injected, or otherwise inserted, within the annulus fibrosis 1704 around the full nucleus implant 1750 and while the partial nucleus implant 1600 cures.

In a particular embodiment, the partial nucleus implant 1600 can be injected, or otherwise installed, using a posterior surgical approach, as shown. Further, the partial nucleus implant 1600 can be injected, or otherwise installed, through a posterior incision 1706 made within the annulus fibrosis 1704 of the intervertebral disc 1700. Alternatively, the partial nucleus implant 1600 can be injected, or otherwise installed, using an anterior surgical approach or a lateral surgical approach. Further, in a particular embodiment, the material used to create the partial nucleus implant 1600 can fill or seal the incision 1706 created within the annulus fibrosis.

Method of Revising a Prior Nucleus Replacement Surgery

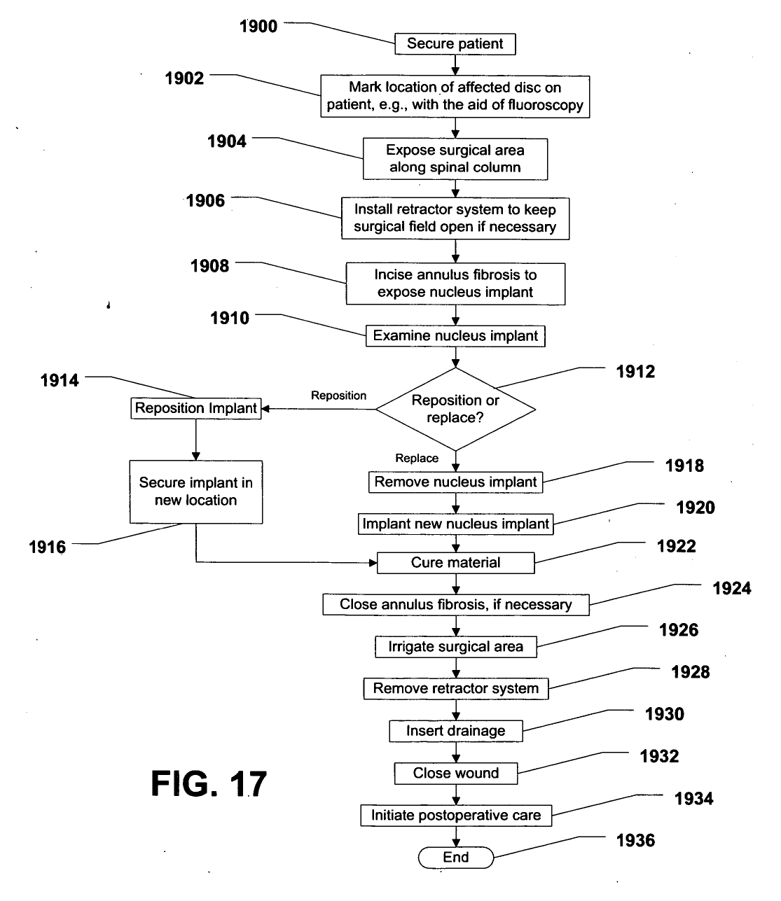

Referring to FIG. 17, a method of revising a prior nucleus replacement surgery is shown and commences at block 1900. At block 1900, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant a partial nucleus implant according to one or more of the embodiments described herein. As such, the patient may be secured in a different position, e.g., in a prone position for a posterior approach or in a lateral decubitus position for a lateral approach.

Moving to block 1902, the location of the affected disc is marked on the patient, e.g., with the aid of fluoroscopy. At block 1904, the surgical area along spinal column is exposed. Further, at block 1906, a surgical retractor system can be installed to keep the surgical field open, if necessary. For example, the surgical retractor system can be a Medtronic Sofamor Danek Endoring™ Surgical Retractor System. In an alternative embodiment, the surgical technique used to access the spinal column may be a “keyhole” technique and a retractor system may not be necessary.

Proceeding to block 1908, the annulus fibrosis of the affected disc is incised to expose the nucleus implant that was implanted during a prior nucleus replacement surgery. At block 1910, the prior nucleus implant is examined. Moving to decision step 1912, the surgeon can determine whether to reposition or replace the prior nucleus implant. For example, the surgeon can make this determination based on the condition of the annulus fibrosis. Also, the surgeon can make this determination based on the condition of the prior nucleus implant.

If the surgeon determines to reposition the implant, the method continues to block 1914 and the implant is repositioned. In a particular embodiment, the surgeon can reposition the implant using a repositioning instrument. For example, the repositioning instrument can be an elongated device that is configured to push, pull, rotate, or otherwise manipulate, the prior implant. Moving to block 1916, the implant is secured in the new location. In a particular embodiment, the implant can be secured in the new location with one or more biocompatible materials. In a particular embodiment, the biocompatible materials can include one or more curable biomaterials. The curable biomaterials can include any natural or synthetic materials with or without adhesive properties that can undergo phase transformation from a flowable to a non-flowable state due to gelation, crystallization, crosslinking, solidification, etc.

The curable biomaterials can include polymer materials, hydrogels, proteins, and polysaccharides. For example, the polymer materials can include polyurethane materials, polyaryletherketone (PAEK) materials, polyolefm materials, silicone materials, silicone polyurethane copolymer materials, polymethylmethacrylate materials, epoxy materials, cyanoacrylate materials, hydrogels, resorbable polymer materials, or a combination thereof The polyaryletherketone (PAEK) materials can include polyetherketone (PEK), polyetheretherketone (PEEK), polyetherketoneketone (PEKK), polyetherketoneetherketoneketone (PEKEKK), or a combination thereof. Further, the polyolefin materials can include polypropylene, polyethylene, halogenated polyolefin, flouropolyolefin, or a combination thereof.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

The proteins can include collagen, silk, elastin, keratin, albumin, gelatin, de-mineralized bone matrix, fibrin, or a combination thereof. Further, the polysaccharides can include glycosaminoglycan (GAG), hyaluronic acid (HA), carboxymethylcellulose (CMC), or a combination thereof.

In a particular embodiment, the material used to secure the prior implant in the new position can be delivered using a device for injection, extrusion, infusion, insertion, or deposition. The device can be a syringe, a double-barrel syringe, a caulk gun, or any other device that can dispense a material via pressure or force.

In an alternative embodiment, the implant can be secured in the new location using a partial nucleus implant, e.g., one of the partial nucleus implants described herein.

Returning to decision step 1912, if the surgeon determines that the prior nucleus implant should be replaced, the method proceeds to block 1918 and the prior nucleus implant is removed. In a particular embodiment, the prior nucleus implant can be removed by cutting the prior nucleus implant into small pieces and retrieving and removing each piece. For example, U.S. patent application Ser. No. 10/976,893, filed on Nov. 1, 2004, and entitled “Devices and Methods for Explanation of Intervertebral Disc Implants,” discloses a device and method that can be used to remove a prior nucleus implant. At block 1920, a new nucleus implant is implanted within the annulus fibrosis. For example, U.S. Pat. No. 6,893,466, entitled “Intervertebral Disc Nucleus Implants and Methods,” discloses a method of implanting a new nucleus implant.

From block 1916 or block 1920, the method proceeds to block 1922 and the material used to secure the prior implant or the material within the new nucleus implant is cured. For example, the material can be cured using an energy source. For example, the energy source can be a light source that emits visible light, infrared (IR) light, or ultra-violet (UV) light. Further, the energy source can be a heating device, a radiation device, or other mechanical device.

Proceeding to block 1924, the annulus fibrosis can be closed, if necessary. In a particular embodiment, the annulus fibrosis can be closed by simply allowing the annulus fibrosis to close naturally. Also, a sealant may be used to facilitate closure of the annulus fibrosis. At block 1926, the intervertebral space can be irrigated. Further, at block 1928, the retractor system can be removed. At block 1930, a drainage, e.g., a retroperitoneal drainage, can be inserted into the wound. Additionally, at block 1932, the surgical wound can be closed. The surgical wound can be closed using sutures, surgical staples, or any other surgical technique well known in the art. Moving to block 1934, postoperative care can be initiated. The method ends at step 1936.

Method of Augmenting a Nucleus Replacement Surgery

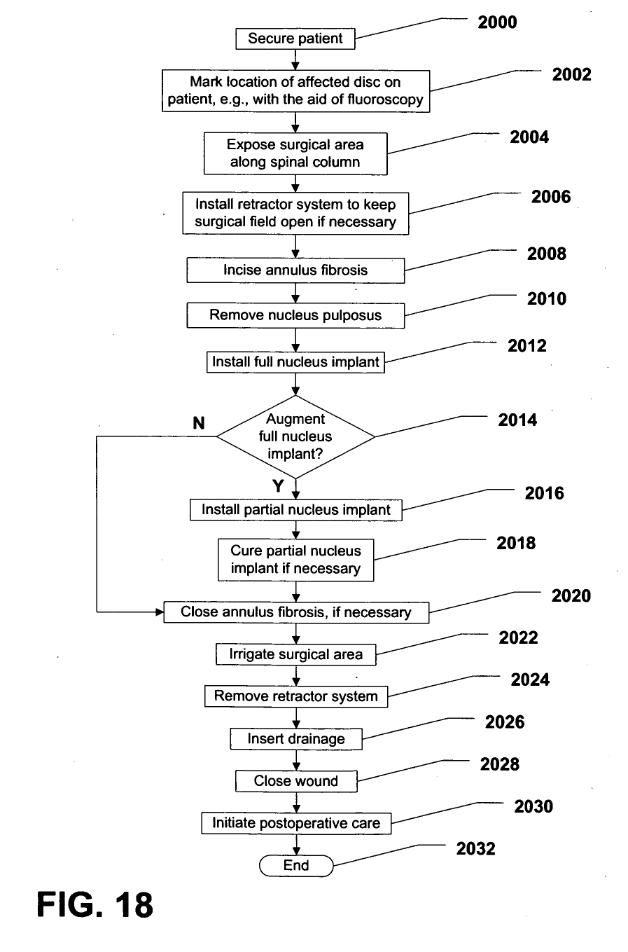

Referring to FIG. 18, a method of augmenting a nucleus replacement surgery is shown and commences at block 2000. At block 2000, a patient is secured on an operating table. For example, the patient can be secured in a supine position to allow an anterior approach to be used to access the patient's spinal column. Further, the patient may be placed in a “French” position in which the patient's legs are spread apart. The “French” position can allow the surgeon to stand between the patient's legs. Further, the “French” position can facilitate proper alignment of the surgical instruments with the patient's spine. In another particular embodiment, the patient can be secured in the supine position on an adjustable surgical table.

In one or more alternative embodiments, a surgeon can use a posterior approach or a lateral approach to implant a partial nucleus implant according to one or more of the embodiments described herein. As such, the patient may be secured in a different position, e.g., in a prone position for a posterior approach or in a lateral decubitus position for a lateral approach.

Moving to block 2002, the location of the affected disc is marked on the patient, e.g., with the aid of fluoroscopy. At block 2004, the surgical area along spinal column is exposed. Further, at block 2006, a surgical retractor system can be installed to keep the surgical field open, if necessary. For example, the surgical retractor system can be a Medtronic Sofamor Danek Endoring™ Surgical Retractor System. In an alternative embodiment, the surgical technique used to access the spinal column may be a “keyhole” technique and a retractor system may not be necessary.

Proceeding to block 2008, the annulus fibrosis of the affected disc is incised to expose the nucleus pulposus within the intervertebral disc. At block 2010, the nucleus pulposus is removed. Moving to block 2012, a full nucleus implant is installed within the intervertebral disc space created by the removal of the nucleus pulposus.

Continuing to decision step 2014, the surgeon can determine whether to augment the full nucleus implant. In a particular embodiment, the surgeon may augment the full nucleus implant if the full nucleus implant does not substantially fill the intervertebral disc space created by the removal of the nucleus pulposus. If the surgeon determines to augment the full nucleus implant, the method continues to block 2016 and a partial nucleus implant can be installed adjacent to the full nucleus implant, e.g., above the full nucleus implant, below the full nucleus implant, partially around the full nucleus implant, completely around the full nucleus implant, or a combination thereof.

In a particular embodiment, the partial nucleus implant can substantially secure the full nucleus implant within the intervertebral disc space. Further, the partial nucleus implant can be made from one or more biocompatible materials. In a particular embodiment, the biocompatible materials can include one or more curable biomaterials. The curable biomaterials can include any natural or synthetic materials with or without adhesive properties that can undergo phase transformation from a flowable to a non-flowable state due to gelation, crystallization, crosslinking, solidification, etc.

The curable biomaterials can include polymer materials, hydrogels, proteins, and polysaccharides. For example, the polymer materials can include polyurethane materials, polyaryletherketone (PAEK) materials, polyolefin materials, silicone materials, silicone polyurethane copolymer materials, polymethylmethacrylate materials, epoxy materials, cyanoacrylate materials, hydrogels, resorbable polymer materials, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof. Further, the polyolefin materials can include polypropylene, polyethylene, halogenated polyolefin, flouropolyolefin, or a combination thereof.

The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof. The resorbable polymers can include polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

The proteins can include collagen, silk, elastin, keratin, albumin, gelatin, de-mineralized bone matrix, fibrin, or a combination thereof. Further, the polysaccharides can include glycosaminoglycan (GAG), hyaluronic acid (HA), carboxymethylcellulose (CMC), or a combination thereof.

After the partial nucleus implant is installed at block 2016, the method proceeds to block 2018. At block 2018, the partial nucleus implant can be cured, if necessary. In other words, if the partial nucleus implant is an expandable implant or an injectable implant, the partial nucleus implant may be cured. For example, the partial nucleus implant can be cured using an energy source. For example, the energy source can be a light source that emits visible light, infrared (IR) light, or ultra-violet (UV) light. Further, the energy source can be a heating device, a radiation device, or other mechanical device.

Proceeding to block 2020, the annulus fibrosis can be closed, if necessary. In a particular embodiment, the annulus fibrosis can be closed by simply allowing the annulus fibrosis to close naturally. Also, a sealant may be used to facilitate closure of the annulus fibrosis. At block 2022, the intervertebral space can be irrigated. Further, at block 2024, the retractor system can be removed. At block 2026, a drainage, e.g., a retroperitoneal drainage, can be inserted into the wound. Additionally, at block 2028, the surgical wound can be closed. The surgical wound can be closed using sutures, surgical staples, or any other surgical technique well known in the art. Moving to block 2030, postoperative care can be initiated. The method ends at step 2032.

Returning to decision step 2014, if the surgeon determines not to augment the full nucleus implant, the method proceeds directly to block 2020 and continues as described herein.

### CONCLUSION

With the configuration of structure described above, the partial nucleus implant according to one or more of the embodiments disclosed herein provides a device that may be implanted to revise a prior nucleus implant surgery. Further, the partial nucleus implant according to one or more of the embodiments described herein provides a device that may be implanted to augment a nucleus implant surgery. For example, the partial nucleus implant can be implanted around, superior to, inferior to, anterior to, posterior to, laterally adjacent to, or otherwise adjacent to a prior full nucleus implant in order to align the full nucleus implant in a proper position and prevent the full nucleus implant from migrating within an annulus fibrosis of an intervertebral disc to a position that is painful, or otherwise problematic, to a patient.

In alternative embodiments, other types of implant surgeries may be revised using similar partial implants. For example, chin implants, cheek implants, calf implants, and other implants that are at risk for migration may be repositioned during a revision surgery and held in place using a partial implant, e.g., an injectable partial implant.

Additionally, other disc space or intervertebral devices may be installed or revised as described herein. These devices can include rigid fusion devices such as those offered by or developed by Medtronic, Inc. of Minneapolis, Minn. under brand names such as INTERFIX cage, INTERFIX RP cage, LT cage, CORNERSTONE spacer, TELAMON spacer, MDII and MDIII threaded bone dowels, PRECISION GRAFT and PERIMETER ring spacers. Additionally, those devices can include prosthetic motion preserving discs such as those offered by or developed by Medtronic, Inc. under brand names such as MAVERICK, BRYAN, PRESTIGE, or PRESTIGE LP. The devices can include single articulating surface motion preserving discs, double articulating surface motion preserving discs, or a combination thereof.

In still another alternative, motion preserving interbody devices can include devices that extend posteriorally from the interbody space and include features for providing posterior motion. In still another alternative, spherical, ellipsoidal, or similarly shaped disc replacement devices may be installed in the interbody space. Further, these devices can include the SATELLITE system offered by or developed by Medtronic, Inc. In still another alternative, a disc replacement device may be an elastically deformable device comprising a resilient or an elastomeric material such as silicone, polyurethane, polyolefin rubber or a resilient polymer, and/or may comprise a mechanical spring component.

Alternatively, interbody motion preserving devices may include nucleus replacement implants that work in conjunction with all or portions of the natural annulus. Such nucleus replacement implants may include those offered by or developed by Medtronic, Inc under a brand name such as NAUTILUS or offered by or developed by Raymedica, Inc. of Minneapolis, Minn. under brand names such as PDN-SOLO and PDN-SOLO XL. Injectable nucleus replacement material including a polymer based system such as DASCOR™ by Disc Dynamics of Eden Prairie, Minn. or a protein polymer system such as NuCore™ Injectable Nucleus by Spine Wave, Inc. of Shelton, Conn. may be alternatives for preserving interbody motion. In a particular embodiment, any of the implant devices described above may be installed or revised as described herein.

The above-disclosed subject matter is to be considered illustrative, and not restrictive, and the appended claims are intended to cover all such modifications, enhancements, and other embodiments that fall within the true spirit and scope of the present invention. For example, it is noted that the expandable components in the fifth embodiment described herein are referred to as “superior” and “inferior” for illustrative purposes only and that one or more of the features described as part of or attached to a respective embodiment may be provided as part of or attached to another embodiment in addition or in the alternative. Thus, to the maximum extent allowed by law, the scope of the present invention is to be determined by the broadest permissible interpretation of the following claims and their equivalents, and shall not be restricted or limited by the foregoing detailed description.

What is claimed is:

1. A partial nucleus implant configured to be installed within an intervertebral disc between an inferior vertebra and a superior vertebra proximate to a previously installed full nucleus implant, the partial nucleus implant comprising:
a component configured to be installed within a void between the full nucleus implant and an annulus fibrosis, wherein the component substantially secures the full nucleus implant in a desired position.

2. The partial nucleus implant of claim 1, wherein the component comprises an expandable component that is expandable from a deflated position to one of a plurality of inflated positions.

3. The partial nucleus implant of claim 2, wherein the component is injected with an injectable biocompatible material.

4. The partial nucleus implant of claim 3, wherein the injectable biocompatible material comprises a polymer material, a ceramic material, a hydrogel, a protein, a polysaccharide, a resorbable polymer, or a combination thereof.

5. The partial nucleus implant of claim 4, wherein the polymer material comprises polyurethane, polyolefin, silicone, silicone polyurethane copolymer, polymethylmethacrylate, epoxy, cyanoacrylate, hydrogel, or a combination thereof.

6. The partial nucleus implant of claim 4, wherein the ceramic material comprises calcium phosphate, hydroxyapatite, calcium sulfate, bioactive glass, or a combination thereof.

7. The partial nucleus implant of claim 4, wherein the hydrogel comprises polyacrylamide (PAAM), poly-N-isopropylacrylamnine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

8. The partial nucleus implant of claim 4, wherein the resorbable polymer comprises polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

9. The partial nucleus implant of claim 4, wherein the protein comprises collagen, silk, elastin, keratin, albumin, gelatin, de-mineralized bone matrix, fibrin, or a combination thereof.

10. The partial nucleus implant of claim 4, wherein the polysaccharide comprises glycosaminoglycan (GAG), hyaluronic acid (HA), carboxymethylcellulose (CMC), or a combination thereof.

11. The partial nucleus implant of claim 1, wherein the component comprises a cured biomaterial.

12. The partial nucleus implant of claim 11, wherein the component is formed in situ.

13. The partial nucleus implant of claim 11, wherein the component is preformed.

14. The partial nucleus implant of claim 13, wherein the component is formed from a biocompatible material.

15. The partial nucleus implant of claim 14, wherein the biocompatible material comprises a polymer material, a hydrogel, a protein, a polysaccharide, or a combination thereof.

16. The partial nucleus implant of claim 15, wherein the polymer material comprises a polyurethane material, a polyolefm material, a polyaryletherketone (PAEK) material, a silicone material, or a combination thereof.

17. The partial nucleus implant of claim 16, wherein the polyolefin material comprises polypropylene, polyethylene, halogenated polyolefin, flouropolyolefm, or a combination thereof.

18. The partial nucleus implant of claim 16, wherein the polyaryletherketone (PAEK) material comprises polyetherketone (PEK), polyetheretherketone (PEEK), polyetherketoneketone (PEKK), polyetherketoneetherketoneketone (PEKEKK), or a combination thereof.

19. The partial nucleus implant of claim 15, wherein the hydrogel comprises polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

20. The partial nucleus implant of claim 15, wherein the protein comprises collagen, silk, elastin, keratin, albumin, gelatin, de-mineralized bone matrix, fibrin, or a combination thereof.

21. The partial nucleus implant of claim 15, wherein the polysaccharide comprises glycosaminoglycan (GAG), hyaluronic acid (HA), carboxymethylcellulose (CMC), or a combination thereof.

22. The partial nucleus implant of claim 1, wherein the component is installed around the full nucleus implant.

23. The partial nucleus implant of claim 1, wherein the component is installed anterior to the full nucleus implant.

24. The partial nucleus implant of claim 1, wherein the component is installed posterior to the full nucleus implant.

25. The partial nucleus implant of claim 1, wherein the component is installed lateral to the full nucleus implant.

26. The partial nucleus implant of claim 1, wherein the component is installed superior to the full nucleus implant.

27. The partial nucleus implant of claim 1, wherein the component is installed inferior to the full nucleus implant.

28. The partial nucleus implant of claim 1, wherein the component comprises a superior component installed superior to the full nucleus implant and an inferior component installed inferior to the full nucleus implant.

29. The partial nucleus implant of claim 28, wherein each component comprises a concave surface configured to engage the full nucleus implant.

30. The partial nucleus implant of claim 29, wherein each component comprises a convex surface configured to engage a vertebra.

31. The partial nucleus implant of claim 20, wherein each component further comprises a plurality of teeth extending from the convex surface.

32. A partial nucleus implant to be installed within an intervertebral disc between an inferior vertebra and a superior vertebra around a previously installed full nucleus implant, the partial nucleus implant comprising:
a superior component having a superior surface configured to engage a superior vertebra and an inferior surface configured to engage a full nucleus implant; and
an inferior component having an inferior surface configured to engage an inferior vertebra and a superior surface configured to engage a full nucleus implant.

33. The partial nucleus implant of claim 32, wherein the superior surface of the superior component and the inferior surface of the inferior component are convex.

34. The partial nucleus implant of claim 33, wherein the inferior surface of the superior component and the superior surface of the inferior component are concave.

35. The partial nucleus implant of claim 34, further comprising a plurality of teeth extending from the superior surface of the superior component.

36. The partial nucleus implant of claim 35, further comprising a plurality of teeth extending from the inferior surface of the inferior component.

37. The partial nucleus implant of claim 32, wherein the superior component and the inferior component are expandable from a deflated position to one of a plurality of inflated positions.

38. The partial nucleus implant of claim 37, wherein the superior component and the inferior component are injected with an injectable biocompatible material.

39. The partial nucleus implant of claim 38, wherein the injectable biocompatible material includes a polymer material, a hydrogel, a protein, a polysaccharide, a resorbable polymer, or a combination thereof.

40. The partial nucleus implant of claim 39, wherein the polymer material comprises a polyurethane material, a polyolefin material, a silicone material, or a combination thereof.

41. The partial nucleus implant of claim 40, wherein the polyolefin material comprises polypropylene, polyethylene, halogenated polyolefm, or flouropolyolefm.

42. The partial nucleus implant of claim 39, wherein the hydrogel comprises polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

43. The partial nucleus implant of claim 39, wherein the resorbable polymer comprises polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

44. The partial nucleus implant of claim 39, wherein the protein comprises collagen, silk, elastin, keratin, albumin, gelatin, de-mineralized bone matrix, fibrin, or a combination thereof.

45. The partial nucleus implant of claim 39, wherein the polysaccharide comprises glycosaminoglycan (GAG), hyaluronic acid (HA), carboxymethylcellulose (CMC), or a combination thereof.

46. A method of revising a prior nucleus replacement surgery, the method comprising:
examining a prior nucleus implant;
examining an annulus fibrosis around the prior nucleus implant; and
determining whether to reposition or replace the prior nucleus implant.

47. The method of claim 46, further comprising:
repositioning the prior nucleus implant; and
substantially securing the prior nucleus implant in a new position.

48. The method of claim 47, wherein the prior nucleus implant is substantially secured in the new position using a curable biomaterial.

49. The method of claim 48, wherein the curable biomaterial includes a polymer material, a hydrogel, a protein, a polysaccharide, a resorbable polymer, or a combination thereof.

50. The method of claim 49, wherein the polymer material is a polyurethane material, a polyolefin material, a polyaryletherketone (PAEK) material, a silicone material, or a combination thereof.

51. The method of claim 50, wherein the polyolefin material is polypropylene, polyethylene, halogenated polyolefin, flouropolyolefin, or a combination thereof.

52. The method of claim 50, wherein the polyether material is polyetherketone (PEK), polyetheretherketone (PEEK), polyetherketoneketone (PEKK), polyetherketoneetherketoneketone (PEKEKK), or a combination thereof.

53. The partial nucleus implant of claim 49, wherein the hydrogel comprises polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

54. The partial nucleus implant of claim 49, wherein the resorbable polymer comprises polylactide (PLA), polyglycolide (PGA), polylactide-co-glycolide (PLG), Poly-e-caprolactone, polydiaoxanone, polyanhydride, trimethylene carbonate, poly-β-hydroxybutyrate (PHB), poly-g-ethyl glutamate, poly-DTH-iminocarbonate, poly-bisphenol-A-iminocarbonate), polyorthoester (POE), polyglycolic lactic acid (PGLA), or a combination thereof.

55. The method of claim 49, wherein the protein comprises collagen, silk, elastin, keratin, albumin, gelatin, de-mineralized bone matrix, fibrin, or a combination thereof.

56. The method of claim 49, wherein the polysaccharide comprises glycosaminoglycan (GAG), hyaluronic acid (HA), carboxymethylcellulose (CMC), or a combination thereof.

57. The method of claim 47, wherein the prior nucleus implant is secured in the new position using a partial nucleus implant.

58. The method of claim 57, wherein the partial nucleus implant includes an expandable component that is injected with an injectable biocompatible material.

59. The method of claim 58, wherein the expandable component is expandable to fill a void between the prior nucleus implant and the annulus fibrosis.

60. The method of claim 59, wherein the expandable component prevents the prior nucleus implant from moving with respect to the annulus fibrosis.

61. A method of revising a prior nucleus replacement surgery, the method comprising:
repositioning a prior nucleus implant; and
substantially securing the prior nucleus implant in a new position.

62. The method of claim 61, wherein the prior nucleus implant is substantially secured in the new position using a partial nucleus implant.

63. A method of installing a nucleus implant, the method comprising:
installing a full nucleus implant; and
installing a partial nucleus implant adjacent to the full nucleus implant.
